(19) United States
(12) Patent Application Publication
(10) Pub. No.: US 2007/0128670 A1
(43) Pub. Date: Jun. 7, 2007

(54) Methods for the identification and preparation of regulator/suppressor t lymphocytes, compositions and use thereof

(75) Inventors: David Klatzmann, Paris (FR); Francois Lemoine, Montrouge (FR)

Correspondence Address: FISH & RICHARDSON PC, P.O. BOX 1022, MINNEAPOLIS, MN, 55440-1022, US

(73) Assignee: ASSISTANCE PUBLIQUE, HOPITAUX DE PARIS, Paris (FR)

(21) Appl. No.: 10/584,728

(22) PCT Filed: Dec. 23, 2004
(86) PCT No.: PCT/FR04/03374

§ 371 Date: Jun. 23, 2006

(30) Foreign Application Priority Data
Dec. 24, 2003 (FR) 0315360

(51) Int. Cl. G01N 33/567; C12N 5/08

(52) U.S. Cl. 435/7.21; 435/372

(57) ABSTRACT

The invention relates to the fields of biology, genetics and medicine. The invention describes methods and compositions enabling (1) the identification of suppressor T cells or lymphocytes (Ts) or the precursors thereof (pTs) for diagnostic or therapeutic purposes and for carrying out genomic or proteomic studies, particularly for the identification of novel markers and/or therapeutic targets for said cells; (2) the production of suppressor T cells or lymphocytes (Ts) or the precursors thereof (pTs) and/or the manipulations thereof in vivo or ex vivo for controlling various pathological conditions, including diseases associated with abnormal activity of effector and/or regulator lymphocytes. The invention relates to the preparation of said compositions based on Ts lymphocytes and pTs, and to the use thereof in cell therapies. The compositions or cell populations based on Ts lymphocytes and pTs obtained according to the invention are particularly suitable for the treatment of tumors, autoimmune diseases, allergies, graft-versus-host disease, graft-versus-infection effects (GVI) or graft-versus-leukemia effects (GVL), inflammatory diseases, type 1 diabetes viral, bacterial or parasitic infections, for immune reconstitution or for induction of tolerance in the event of transplantation of stem cells, tissues or organs in a mammal.

The invention relates to the fields of biology, genetics and medicine. The invention describes methods and compositions enabling the identification, production and manipulation ex vivo and in vivo of suppressor T cells or lymphocytes (Ts), including the precursors thereof (pTs), also called regulator T cells (or Treg), and the use of said suppressor lymphocytes for controlling various pathological conditions, including diseases associated with abnormal activity of effector and/or regulator/suppressor T lymphocytes. The invention relates to the preparation of said compositions based on Ts lymphocytes and pTs, and to the use thereof in cell and/or gene therapies. The compositions or cell populations based on Ts lymphocytes and pTs obtained according to the invention are particularly suitable for the treatment of genetic or acquired diseases, particularly tumors, autoimmune diseases, allergies, graft-versus-host disease, graft-versus-infection effects (GVI) or graft-versus-leukemia effects (GVL), inflammatory diseases including for example atherosclerosis, diabetes, viral, bacterial or parasitic infections, for immune reconstitution or induction of tolerance in the event of transplantation of stem cells, tissues or organs in mammals.

The existence in the immune system of cells capable of carrying out regulator/suppressor functions had long been suspected. In the 1980s, a number of scientific publications revealed the existence of suppressor activities within the T lymphocyte population. However, the impossibility of characterizing and isolating cells with said function from the total lymphocyte population, which also has many other functions including in particular effector functions, precluded a better understanding of this phenomenon. In 1995, a subpopulation of CD4+ T lymphocytes constitutively expressing the CD25 marker was identified in rodents as playing a major role in controlling the immune response and autoimmune diseases. Said CD4+/CD25+ T cells, also called regulator or suppressor T cells (Ts), account for approximately 5-10% of CD4+ T cells in the mouse. Ts cells express an antigen-specific T cell receptor, like other T lymphocytes, but their global action is partially nonspecific with the possibility of recruiting other additional suppressor T lymphocytes through a phenomenon called “infectious suppression”. In humans, a CD4+/CD25+ regulatory cell population, representing less than 5% of CD4+ T cells, has also been described.

Several experiments have now clearly established the therapeutic potential of CD4+/CD25+ suppressor T lymphocytes in numerous diseases.

For instance, Ts cells play a major role in controlling autoimmune diseases like type 1 diabetes or graft-versus-host disease (GVHD) induced by allogeneic T lymphocytes. Addition of Ts cells to grafts containing allogeneic hematopoietic stem cells and effector T lymphocytes can control the onset or emergence of GVHD. Injection of Ts cells can attenuate the autoimmune response in autoimmune polymyositis (unpublished). Ts cells also play a major role in the establishment or induction of tolerance during tissue or organ transplantation and/or in the presence of immunogenic molecules such as transgenes. Ts cells further play an important role in modulating the response to infectious agents, and particularly to intracellular bacteria and viruses.

Ts cells play a role in several inflammatory diseases such as atherosclerosis. In this case, an absence or a reduction in the number of Ts cells leads to an acceleration of disease development and an increase in disease severity (unpublished results).

It is now well established that Ts cells prevent the development of effector anti-tumor responses, which otherwise can lead to tumor eradication. In mice, Ts cell depletion leads in many cancer models to tumor eradication through an effector immune response. In humans, a correlation between an unfavorable disease course and Ts cells has been described in several malignant pathologies. Tumor Ts cells are associated with decreased survival. In addition, pharmacologic modulation of Ts cells improves treatments based on Tumor Infiltrating Lymphocytes.

Ts cells are also important in vaccination since they can suppress the development of a specific immune response. Likewise, Ts depletion or reduction very markedly improves the effects of an anticancer vaccine.

Lastly, many publications now report the presence of an abnormal number or percentage of Ts cells in various diseases, and during the progression of a given disease.

All of these arguments indicate that the identification, selection, expansion or depletion of CD4+/CD25+ regulator T cells in vitro or in vivo represent an enormous diagnostic and therapeutic potential for many diseases and in particular for autoimmune diseases, inflammatory diseases, infectious diseases, cancer and graft rejection.

The characterization of Ts cells is also of major importance. While there are few data on the homeostasis and regulation of this Ts lymphocyte population, it appears that the Foxp3 transcription factor is an important player in the development and function of CD4+/CD25+ suppressor T lymphocytes. It has not been established that Foxp3 is expressed on all Ts cells but the absence of Foxp3 expression in mice is correlated with a dramatic loss of Ts cell function, whereas forced expression of Foxp3 in effector T lymphocytes converts them to Ts cells.

Although the CD4 and CD25 markers characterize a cell population that contains suppressor T lymphocytes, it appears in fact that the suppressor functions are not entirely due to CD4+/CD25+ cells and above all that not all CD4+/CD25+ are suppressor cells. In fact, the CD25 marker is also expressed by activated effector T cells. The identification and purification of Ts cells on the basis of said marker is a major problem due to the risk that what is actually identified and purified will be activated effector T cells. In the context of a given immunologic disorder, activated T lymphocytes expressing CD4 and CD25 have a high probability of containing precisely those effector T cells against which a therapeutic intervention is desirable. Thus the use of CD4 and CD25 in a diagnostic context (identification) would not be reliable, and in a therapeutic context (purification, injection) would run the risk of being ineffective or even exacerbating the disease.

The best marker currently known to be capable of differentiating Ts cells from activated effector T lymphocytes is the expression of the Foxp3 transcription factor. However, this intracellular transcription factor cannot be used in simple methods of immunophenotypic identification and purification. Other markers like CD62L allow a better characterization of Ts cells but are far from enabling a perfect identification. Moreover, several publications have demonstrated the existence of suppressor activities within the CD4+/CD25− population and in certain CD8+ cells. It therefore appears that the diagnostic and therapeutic use of Ts cells clearly depends on the specific identification thereof and that current knowledge has so far not revealed any marker specific of suppressor T lymphocytes.

Furthermore, while some Ts lymphocytes appear to differentiate in thymus (they are often called “natural” Ts cells), other Ts lymphocytes might be generated peripherally and nothing is known about the ontogenic development of Ts cells from T cell progenitors.

The invention provides for the first time the opportunity to identify, isolate, analyze (transcriptome, proteome, etc.) and manipulate (culture, activation, depletion, genetic modifications, etc.) suppressor T cell populations, particularly human, and in particular (i) populations of Ts precursors and (ii) populations of pure Ts cells among CD4+ and CD8+ cells. The invention derives from the discovery that the CD90 molecule, also called THY-1, represents a marker which is characteristic of human CD4+ and/or CD8+ Ts cells, and the precursors thereof, and can be efficiently used to identify said cell populations.

The THY-1 antigen (Seki et al., 1985; Planelles et al., 1995) corresponds to a well characterized surface glycoprotein anchored to the membrane by a phosphatidylinositol bridge. Said protein belongs to the immunoglobulin superfamily and contains approximately 140 amino acids (25-30 kDa). This antigen was initially identified as a differentiation marker expressed in mouse thymus and brain. In humans, THY-1 is expressed on a small percentage of fetal thymocytes, on immature CD34+hematopoietic progenitors and on less than 1% of CD3⁺ lymphocytes present in the peripheral circulation. THY-1 is also expressed on mesenchymal cells, endothelial cells and in several established cell lines. The function of THY-1 is not known.

In mice, THY-1 is expressed in thymus on T cell precursors and progenitors. It is also expressed on regulatory cells (Mukasa et al., Clin. Exp. Immunol. 96 (1994) 138; Torre-Amione et al., Cell. Immunol. 124 (1989) 50; Sakatsume et al., Int. Immunol. 3 (1991) 377) as well as on all circulating T lymphocytes. For this reason, it cannot be a discrimatory marker for a particular cell type. Moreover, two isoforms Thy-1.1 and Thy-1.2 have been described in mice.

The inventors have now discovered that, in a surprising manner, the expression of the THY-1 molecule in humans is closely correlated with Ts activity, and that the THY-1 molecule is a marker specific of suppressor T lymphocytes, enabling in particular the identification, selection, expansion or depletion in vitro or in vivo of Ts precursors and/or pure Ts populations among CD4+ or CD8+ lymphocytes.

A first aspect of the invention therefore relates to a method for obtaining, preparing or producing suppressor T lymphocytes (and/or the precursors thereof), comprising a step of selection, separation, and/or isolation of T lymphocytes expressing the THY-1 molecule. Said step can be carried out on any biological samples comprising lymphocytes.

A more particular object of the invention relates to a method for obtaining, preparing or producing suppressor T lymphocytes (and/or the precursors thereof) comprising:

(a) obtaining a population of mammalian cells comprising T lymphocytes, and

(b) recovering T lymphocytes expressing the THY-1 antigen.

T lymphocytes expressing the THY-1 antigen are preferably selected, separated, isolated, recovered or eliminated by means of a ligand specific of THY-1. Advantageously, the ligand is selected in the group consisting of an antibody or an antibody fragment. For example, the ligand can be immobilized on a support or placed in solution. Such ligand is more fully defined in the description which follows from the invention. In addition, step (b) can be preceded and/or followed by a step of amplification of T lymphocytes and/or a step of purification of lymphocyte subpopulation(s), such as for example CD4+ or CD8+ lymphocytes, or lymphocytes specific of a given antigen.

Another object of the invention relates to a method for the identification and/or quantification of suppressor T lymphocytes (and/or the precursors thereof) in a cell population, comprising exposing said cell population to a ligand specific of THY-1 and determining and/or quantifying the formation of a complex between the ligand and the cells, formation of said complexes indicating the presence and/or the quantity of suppressor T lymphocytes (and/or the precursors thereof) in the cell population. The cells that bind the ligand can be separated from cells that do not bind the ligand.

Another object of the invention relates to the use of a ligand specific of the THY-1 antigen for the enrichment or depletion ex vivo of suppressor T lymphocytes (and/or the precursors thereof) in a cell population. The THY-1 antigen itself can be used as marker for selection of Ts lymphocytes or pTs within a cell population. Another object of the invention is based on a method of diagnosis in a patient, comprising determining the presence, the number or the state of activity of Ts cells in said patient by using a ligand specific of THY-1. Said diagnosis can be carried out in vitro, ex vivo or in vivo, and enables the detection of a pathological condition related to the activity of the immune system, or the monitoring of the efficacy of a treatment, or the selection of a patient in view of being included in a particular therapeutic protocol.

Another object of the invention is based on the use of a ligand specific of the THY-1 antigen for the selection, identification, sorting or preparation (in vitro or ex vivo) of Ts lymphocytes or pTs.

The invention further relates to suppressor T lymphocytes (and/or the precursors thereof) expressing the THY-1 antigen that can be obtained through an inventive method.

Another object of the invention is based on the use of a ligand specific of the THY-1 antigen for preparing a diagnostic composition intended for the selection, identification or quantification in vivo of suppressor T lymphocytes (including the precursors thereof).

Another object of the invention is based on the use of a ligand specific of the THY-1 antigen for preparing a therapeutic composition intended for the modification, stimulation or elimination in vivo of suppressor T lymphocytes. In this respect, a particular object of the invention relates to the use of a ligand specific of THY-1 for enriching or depleting suppressor T lymphocytes (including the precursors thereof) ex vivo or in vivo in a cell population.

Another aspect of the invention is based on the use of the THY-1 antigen as selection marker for the enrichment or depletion, in vivo, in vitro or ex vivo, of Ts lymphocytes or pTs in a cell population.

The invention also relates to suppressor T lymphocytes (including the precursors thereof) expressing the THY-1 antigen that can be obtained by a method such as defined hereinabove, and a population of cells enriched in Ts cells or pTs, in which at least 30%, preferably at least 50%, even more preferably at least 65% of the T cells express the THY-1 antigen. Cell populations or compositions especially preferred according to the invention comprise at least 75%, preferably at least 80%, of Ts cells or pTs expressing THY-1, more preferably at least 85, 90 or 95%.

The invention further relates to an isolated human T lymphocyte, characterized in that it displays a suppressor activity and in that it expresses the markers CD8 or CD4 and THY-1, as well as a cell population comprising CD8+/THY-1+ or CD4+/THY-1+ suppressor T cells, preferably a population comprising at least 50, 60, 70, 80, 85, 90 or 95% of CD8+/THY-1+ T cells. Said cells can also partly express the CD25 antigen.

In a particular embodiment of the invention, the T lymphocytes present in the mammalian cell population or the Ts lymphocytes or pTs (carrying the THY-1 marker) can be genetically modified so as to express biological products of interest, allowing in particular to improve the efficacy and/or safety of same.

The invention also relates to a pharmaceutical composition comprising cells or cell populations such as defined hereinabove, typically in association with a pharmaceutically acceptable vehicle or excipient.

Another particular object of the invention concerns a pharmaceutical composition comprising human suppressor T cells (and/or the precursors thereof) amplified ex vivo and a pharmaceutically acceptable adjuvant or medium, said amplified cells being enriched in cells expressing the THY-1 antigen and, optionally, in cells specific of a particular antigen, such as allergens, auto-antigens, allo-antigens or antigens of infectious agents. In a preferred manner, the antigen is involved in or specific of a pathological condition selected in the group consisting of an immune disease, in particular autoimmune diseases, inflammatory diseases, graft-versus-host disease, an allergy or graft rejection.

The invention further relates to the preparation of a composition composed of at least one such suppressor T lymphocyte, a population enriched in Ts cells and/or pTs such as defined hereinabove or on the contrary a population depleted of Ts cells and/or pTs and a pharmaceutically acceptable adjuvant or medium as well as the composition itself intended for the carrying out of a therapeutic method.

A particular object of the invention thus also concerns a method for producing a pharmaceutical composition, comprising:
(a) obtaining a biological sample comprising T lymphocytes,
(b) selecting T lymphocytes expressing the THY-1 antigen within said biological sample, and
(c) conditioning said T lymphocytes expressing the THY-1 antigen in a pharmaceutically acceptable adjuvant or medium.

Another particular object of the invention also relates to a method for producing a pharmaceutical composition, comprising:
(a) obtaining a biological sample comprising T lymphocytes,
(b) depleting T lymphocytes expressing the THY-1 antigen from said biological sample, and
(c) conditioning said T lymphocytes not expressing the THY-1 antigen in a pharmaceutically acceptable adjuvant or medium.

The invention also relates to a kit for the isolation or characterization of Ts cells comprising a ligand specific of THY-1, optionally deposited on a support or placed in solution and, optionally, reagents for the detection of the ligand. The ligand is typically placed in a container, such as a plate, syringe, tube, pipette, vial, etc. Said kit can also be used to diagnose the presence of said Ts cells and pTs in a biological sample taken from an individual to be tested or directly in vivo.

The invention further relates to a kit or a composition intended for the elimination of Ts cells and pTs in vivo, in vitro or ex vivo, comprising a ligand specific of THY-1, optionally placed in solution or on a support, and coupled with a toxic product (radioactive, toxins, etc.). The invention is also directed to the use of a Thy-1 ligand to specifically target a viral or non-viral vector to Ts and pTs so as to express genes.

The invention further relates to a kit or a composition to activate Ts cells and pTs in vivo, ex vivo or in vitro, comprising a ligand specific of THY-1, optionally placed in solution or on a support, and coupled with product capable of activating T lymphocytes (for example a cytokine, such as IL-2, IL-7, IL-10, IL-15). The invention is also directed to the use of a ligand of THY-1 to specifically target a viral or non-viral vector to Ts so as to express activator genes or any therapeutic genes.

The Ts cells, the compositions containing isolated or amplified Ts cells and pTs and the compositions enriched in Ts cells and pTs obtained in the context of the invention can advantageously be used for experimental or therapeutic purposes. The cells used in the context of the invention are mammalian cells, typically human. The invention can also be used in particular in primates, and therefore also concerns suppressor T cells from primates, particularly monkey.

A particular object of the invention thus also relates to methods by which to analyze and obtain gene sequences specifically expressed in suppressor T lymphocytes (or the precursors thereof), one method comprising isolating RNA from a population of T lymphocytes expressing the THY-1 antigen, comparing said RNA with RNA extracted from a population of non-suppressor T lymphocytes and recovering RNA specific of suppressor T lymphocytes. The invention also relates to a method such as described hereinabove also comprising the production of a probe from RNA specific of suppressor T lymphocytes (or the precursors thereof) and the screening of a nucleic acid population intended to be hybridized with said probe. One method also corresponds to transcriptome analysis by RNA hybridization on biochips so as to establish expression profiles. These different methods lead to the characterization of the expression of genes important for the differentiation, maturation, regulation and function of Ts and pTs, thereby allowing to define potential new markers and/or therapeutic targets.

A particular object of the invention therefore relates to a method for obtaining proteins specifically expressed in suppressor T lymphocytes (or the precursors thereof). One method comprises isolating proteins from a T lymphocyte population expressing the THY-1 antigen, comparing said proteins with those extracted from a population of non-suppressor T lymphocytes. These different methods lead to the characterization of the expression of proteins important for the differentiation, maturation, regulation and function of Ts and pTs, thereby allowing to define potential new markers and/or therapeutic targets.

A particular object of the invention therefore relates to a method for identifying novel molecules specifically expressed in suppressor T lymphocytes (or the precursors thereof) by immunization with Ts lymphocytes (or pTs) expressing the THY-1 antigen, or cell or protein fractions from said same cells.

Another particular object of the invention also relates to the use, in a therapeutic context, of Ts cells (or pTs), of compositions composed of isolated or amplified Ts cells (or pTs), and of compositions enriched in Ts cells (or pTs) obtained in the context of the invention, for example for the treatment of many subjects, for example human patients suffering from or presenting a risk of developing an immune disease, in particular a disease induced by an abnormal T cell response. The Ts cells (or pTs) are thus suitable for treating various pathologies or diseases induced by a disorder affecting T lymphocytes and in particular a tumor, autoimmune disease, allergy, graft-versus-host disease, inflammatory disease, type 1 diabetes, viral or bacterial infection, and the like. They also promote immune reconstitution and induction of tolerance in the event of engraftment or transplantation of stem cells, tissues or organs in a mammal. This is the case, for example, following bone marrow or hematopoietic stem cell transplantation. The treatment can be preventive or curative. It can also be combined with other treatments.

Human Suppressor T cells (or the Precursors Thereof)

In the context of the invention, the term suppressor T lymphocytes (or cells) denotes a population of T cells characterized by their ability to suppress or diminish immune reactions mediated by effector T cells, such as CD4+ or CD8+ T cells. Said term includes conventional Ts cells, which strongly express the CD25 marker, and the precursors thereof, called pTs, which exhibit suppressor activity and which, in culture, can give rise to conventional Ts cells. In fact, the invention demonstrates the existence of a population of suppressor T cells, denoted pTs, expressing the THY-1 and CD25 markers, exhibiting the suppressor property, and able to give rise in culture to conventional Ts cells. The term suppressor T lymphocytes also includes Ts lymphocytes arising from total (or CD25−) lymphocyte populations, of the type CD4+ or CD8+, expressing THY-1.

As noted in the introduction, while the CD4 and CD25 markers characterize suppressor T lymphocytes (or the precursors thereof) from an immunophenotypic standpoint, in fact it appears that the suppressor functions are not entirely carried by CD4+/CD25+ cells and that not all CD4+/CD25+ cells are suppressor cells. In fact, CD25 is a marker which is also expressed by activated effector T cells. The invention results from the demonstration that the antigenic molecule THY-1 represents a marker characteristic of human Ts cells and pTs and can be efficiently used to identify said cell population.

As indicated earlier, the invention thus relates to a method for obtaining, preparing, selecting or producing human suppressor T lymphocytes (including the precursors thereof) comprising:
(a) obtaining a population of human cells comprising T lymphocytes, and
(b) recovering T lymphocytes expressing THY-1.

In step (a), the cell population can be obtained from biological samples comprising lymphocytes, particularly samples of a tissue selected in the group consisting of bone marrow, spleen, liver, thymus, blood previously or not enriched in T lymphocytes, umbilical cord blood, fetal, newborn or adult peripheral blood, plasma, a lymph node, a tumor, a site of inflammation, a transplanted organ or a cell culture established with one or another of said tissues. The lymphocytes are typically isolated or collected from peripheral blood.

The T lymphocytes expressing THY-1 can be recovered, selected, isolated, depleted or sorted, particularly during step (b), with the help of any ligands specific of THY-1, that is to say, typically any molecules capable of selectively binding Thy-1 at the surface of a cell. The ligand is preferably selected in the group consisting of an antibody, preferably an anti-THY-1 antibody, an analog or fragment of same.

THY-1 is a molecule devoid of an intracytoplasmic domain which interacts with the cell membrane by means of a glycophosphatidylinositol (GPI) which attaches to the membrane through its C-terminal end. The sequence of Thy-1 has been determined and can be found in the literature, such as for example the nucleotide sequence (No. NM 006288 (gi: 199 233 61)) and the amino acid sequence (No. NP 006279 (gi: 199 233 62)) of the human protein. A specific ligand according to the invention is preferably a molecule with the ability to selectively bind a polypeptide comprising all or part of the sequence of the human Thy-1 protein, preferably a molecule comprising an epitope of the human Thy-1 protein. Said ligands are naturally selected in the group consisting of molecules known and/or capable of interacting with the extracellular part of THY-1.

A preferred ligand of THY-1, that can be used in the invention, is an anti-THY-1 antibody (that is to say, an antibody specific of THY-1). The antibody can be polyclonal or monoclonal. It can also be fragments or derivatives of an antibody fragment or derivative displaying substantially the same antigenic specificity, particularly antibody fragments (e.g., Fab, Fab′2, CDRs), humanized antibodies, human antibodies, polyfunctional, monocatenary antibodies (ScFv), or multimeric antibodies (C4bp coupling for example), etc. The antibodies, and therefore the sites of recognition of the THY-1 molecule that can be used to generate a specific ligand, can be produced by conventional methods, comprising immunizing a non-human animal with a THY-1 polypeptide or a fragment of same containing an epitope, and recovering the serum (polyclonal) or spleen cells (so as to produce hybridomas by fusion with suitable cell lines). Various methods for producing polyclonal antibodies from different species have been described in the prior art. Typically, the antigen is combined with an adjuvant (for example Freund's adjuvant) and administered to an animal, for example by subcutaneous injection. Repeated injections may be given. Blood samples are collected and the immunoglobulin and serum are separated.

Classical methods of monoclonal antibody production comprise immunizing a non-human animal with an antigen, and recovering spleen cells which are then fused with immortalized cells, such as myeloma cells. The resulting hybridomas produce monoclonal antibody and can be selected by limit dilutions so as to isolate individual clones. Fab or F(ab′)2 fragments can be produced by digestion with a protease according to conventional techniques.

Preferred antibodies are antibodies specific of the THY-1 protein, that is, having a higher affinity for said protein than for other antigens, although a non-specific and lower affinity binding cannot be excluded. In particular, the term “specific” or “selective” indicates that binding of the ligand to the THY-1 protein can be differentiated from an eventual binding of the ligand to other molecules.

Ts cells and pTs can thus be isolated, in the context of step (b), by contacting the cell population with specific ligands, such as defined hereinabove. Particular examples of specific ligands according to the invention are in particular monoclonal antibodies produced by the hybridomas K17 (ATCC No. HB-8553), clones 5E10, F15-42-1, Thy-1/310, FIB1 (clone AS02), as well as any fragments or derivatives of said antibodies.

Other specific ligands according to the invention are for example artificial ligands, displaying a particular affinity for THY-1. Said ligands can be of different natures, such as nucleic acids (for example aptamers) or synthetic chemical molecules. Such molecules can be generated for example based on the sequences of the sites of recognition of the THY-1 molecule by the specific antibodies defined hereinabove.

Ts cells and pTs can thus be isolated, in the context of step (b), by contacting the cell population with one or more specific ligands, such as those defined hereinabove.

In the scope of the invention, it is possible to use one or more ligands specific of THY-1, possibly in combination with other ligands specific of other T cell markers, such as CD25 in particular. Thus, in a particular embodiment, the invention uses a combination of a THY-1-specific ligand and a CD25-specific ligand. The second ligand can be specific of any other T cell marker, particularly of suppressor T cells, for example markers identified by the genomic and proteomic methods described herein.

The ligand(s) can be immobilized on a support, for example a column or bead (particularly a magnetic bead), or placed in solution. In addition, or as a variant, the ligand can optionally be labelled. Labelling can be carried out by means of a fluorescent, radioactive, luminescent, phosphorescent, chemical or enzymatic label. The detection label is preferably selected in the group consisting of fluorescein, Texas red, rhodamine, phycoerythrin, allophycocyanin, biotin and streptavidin, cyanin.

The complexes formed by the ligand and the labelled cells can then be used to visualize, detect, quantify, sort, isolate and/or deplete the cells, according to various methods known to those skilled in the art. Thus, the cells can be recovered, selected, sorted, separated, isolated, depleted for example by a method selected from among flow cytometry, affinity chromatography, FACS (fluorescent activated cell sorting), MACS (magnetic bead cell sorting), D/MACS (double magnetic bead cell sorting), affinity chromatography (double magnetic bead cell sorting), a selection method on a solid surface (panning), an ELISA test, an RIA test, and the like.

The MACS procedure is described in detail by Miltenyi et al., “High Gradient Magnetic Cell Separation with MACS,” Cytometry 11: 231-238 (1990). To recover the cells, the cells labelled with magnetic beads pass through a paramagnetic separating column. The separating column is placed next to a magnet, thereby creating a magnetic field inside the column. The magnetically labelled cells are trapped in the column, the other cells pass through it. The cells trapped in the column are then eluted.

In the D/MACS procedure, a cell sample is labelled with magnetic beads comprising an antibody, and the cells are harvested or sorted by applying a magnetic field.

According to a preferred embodiment, the cells (for example from peripheral blood) are incubated sequentially with saturating amounts of functionalized anti-THY-1 antibody (e.g., biotin-labelled) and with a solid support (for example microbeads) which has been functionalized (e.g., coated with streptavidin). The cells are then purified by recovering the support, e.g., by magnetic separation of the cells. To enhance purification of the cells, the cells from the positive fraction can subsequently be separated on another column. Purification is generally carried out in a phosphate buffer, although other suitable media can be used.

The cells can be cultured or maintained in any suitable buffer or medium, such as a saline solution, buffer, culture medium, in particular DMEM, RPMI, etc. The cells can be frozen or kept in the cold. They can be formulated in any suitable device or apparatus, such as a tube, flask, ampoule, plate, syringe, bag, and the like, preferably in sterile conditions suited for pharmaceutical use.

As noted earlier, step (b) of the method described hereinabove can advantageously be preceded and/or followed by a step of purification of a T lymphocyte subpopulation (CD4+ and/or CD8+ for example) and/or a lymphocyte amplification step (which can be carried out ex vivo or in vitro).

Amplification can be achieved by activation of the lymphocytes. Said activation can be non-specific (obtained for example by anti-CD3 and/or anti-CD28 antibodies, with or in the presence of an interleukin, for example IL-2) or specific (obtained by antigens or alloantigens presented in an adequate manner to Ts lymphocytes or pTs, for example by antigen-presenting cells (dendritic cells, B lymphocytes, monocytes macrophages, genetically modified cells capable of presenting antigen and activating lymphocytes), exosomes, dexosomes, artificial structures, etc.). The amplification step makes it possible to increase the number of T lymphocytes present in the initial T lymphocyte population (which comprises effector T lymphocytes and suppressor T lymphocytes) before going on to select Ts lymphocytes and pTs, and/or to increase the number of Ts lymphocytes and pTs after having selected T lymphocytes expressing the THY-1 antigen. It is also possible to carry out two amplification steps, one concerning the general T lymphocyte population present in the mammalian cell population, the other concerning the population of Ts lymphocytes and pTs.

In a preferred embodiment, the purification step is carried out in conditions which are favorable to Ts (or pTs), thereby enabling the enrichment thereof. For instance, the invention shows that culture in the absence of N-acetyl cysteine promotes the proliferation of Ts (FIG. 1). In a particular embodiment of the invention, the cells are amplified by culturing them in a medium free of N-acetyl cysteine. Furthermore, the use of certain populations of natural or modified (in particular genetically) antigen-presenting cells can promote the proliferation of Ts (or pTs). For instance, the examples show that dendritic cells derived from CD34+ hematopoietic progenitors and having a phenotype of the interstitial DC type promote the proliferation of Ts (or pTs) (FIG. 2). In a particular embodiment, the cells are amplified by culturing them in the presence of dendritic cells, particularly interstitial dendritic cells.

The population obtained at the end of step (a) can also be enriched in T cells belonging to the general T cell population, i.e., comprising effector T lymphocytes and suppressor T lymphocytes or the precursors thereof.

The population from step (a) can thus be enriched in T cells, possibly in one or more lymphocyte-specific subpopulations (for example CD4+ and/or CD8+). It can also be depleted of certain lymphocyte subpopulations, as the case may be. The population obtained at the end of step (a), which is optionally amplified and/or sorted, thus comprises preferably at least 30%, preferably at least 50%, even more preferably at least 65% of T cells. Particularly preferred compositions enriched in T cells that can be used in step (b) comprise at least 75%, preferably at least 80% of T cells.

The T lymphocyte population expressing the THY-1 antigen can also be amplified. Furthermore, as indicated earlier, it is possible to carry out two amplification steps, one concerning the general T lymphocyte population, the other concerning the population of Ts lymphocytes or pTs.

Thus, a particular object of the invention relates to a method for obtaining suppressor T lymphocytes (and/or the precursors thereof) comprising:
(a) obtaining a mammalian cell population comprising T lymphocytes,
(a′) amplifying the T lymphocytes within said cell population, and
(b) recovering T lymphocytes expressing the THY-1 antigen.

Another particular object of the invention relates to a method for obtaining suppressor T lymphocytes (and/or the precursors thereof) comprising:
(a) obtaining a mammalian cell population comprising T lymphocytes,
(b) recovering T lymphocytes expressing the THY-1 antigen, and
(b′) amplifying said T lymphocytes expressing the THY-1 antigen.

Amplification of lymphocytes belonging to the general T lymphocyte population (effector T cells, Ts cells and pTs) is preferably carried out by culturing the cells in the presence of a cytokine and possibly a stimulating agent. In the case of lymphocytes expressing the THY-1 antigen (Ts lymphocytes and pTs), the culture is continued for a period sufficient to achieve amplification of said cell population within the populations of CD4+ and/or CD8+ T lymphocytes. Activation generally requires culturing the cells in the presence of a cytokine, such as for example interleukin-2 (IL-2), interleukin-7 (IL-7), interleukin-10 (IL-10) or interleukin-15 (IL-15), preferably of human origin. The stimulating agent can be an antigen-presenting cell (APC), i.e., any antigen-presenting cell or any cell promoting activation of T cells, in particular of Ts cells. The APC are preferably irradiated prior to use to avoid the amplification thereof. The APC can be cells isolated from a donor or from the patient himself. They can be selected so as to produce Ts cells and pTs displaying a desired activity profile. Typical examples of such APC include peripheral blood mononuclear cells, dendritic cells, splenocytes, umbilical cord blood cells, tissue or organ samples, and the like. Other suitable Ts or pTs stimulating agents include MHC polymers, lectins (such as PHA), antibodies (such as anti-CD3 and/or anti-CD28 antibodies) or fragments of same, auto-antigens (including tissues, cells, cell fragments or debris, purified peptides or polypeptides, etc., preferably in combination with the APC), etc.

Depending on the envisioned use, the Ts cells and pTs can be amplified in different ways, whether they be antigen-specific or not. In particular, for some uses, large quantities of the complete T cell repertoire are preferably used (e.g., injected). In particular, this method is adapted to patients with a global deficit (quantitative or functional) of Ts cells and pTs. In such indications, Ts cells and pTs are preferably amplified for example, with the help of autologous APC cells and PHA or anti-CD3 and/or anti-CD25 antibodies (or any other T or Ts cell activator) in the presence of cytokines which are the same or different in nature.

Generally, it is important to take into account the specificity of the Ts cells and pTs. In fact, while it is possible to use non-specific Ts cells to control specific immune responses, the use of specific Ts cells appears more efficient. Thus, in humans and mice, Ts lymphocytes and pTs can be grown and amplified in vitro in the presence of a culture medium containing interleukin-2, anti-CD3 and anti-CD28 antibodies. Specific Ts lymphocytes and pTs can also be isolated, generated for example by stimulation in the presence of allogeneic antigen-presenting cells, followed by culture with interleukin 2. According to another embodiment, a more specific amplification can be envisioned, particularly when elimination of specific effector T cells is desired, such as in the context of autoimmune diseases, allergies, graft rejection, GVHD, etc. In such indications, the cells are preferably amplified in the presence of APC presenting particular antigens, for example allogeneic or of infectious origin, so as to promote the amplification of Ts cells preferentially activated against the pathogenic effector T cells. The antigens are presented in the form of peptides or after RNA or DNA transfer.

For the treatment of autoimmune diseases, the Ts cells and pTs preferably come from the patient and are stimulated by autologous APC and auto-antigens from the target tissue, in the presence of cytokines. The auto-antigens can be tissues, cells, cell fragments, purified proteins, peptides, nucleic acids, and the like.

For the treatment of allografts or xenografts, the Ts cells preferably come from the patient and are stimulated by APC or tissues from the donor, in the presence of cytokines. Ts cells from the patient can also be stimulated by autologous APC in the presence of tissues, cells, cell fragments, purified proteins or peptides from the donor and cytokines.

For the treatment of allergies, the Ts cells typically come from the patient and are activated by APC and allergens, in the presence of cytokines.

As indicated earlier, the cytokines which are preferably used are IL-2, IL-10 and/or IL-15.

As indicated earlier, the Ts cells and pTs used to treat various pathologies such as rejection of a transplanted organ, autoimmune diseases, allergies, viral diseases, etc., are preferably autologous, i.e., they come from the subject to be treated. Syngeneic cells can also be used. In other situations, for example in the context of treatment of GVHD or other pathologies, the Ts cells and pTs are typically allogeneic, i.e., they come from a different human being. In these cases, it is preferable to use Ts cells and pTs from a donor subject (e.g., the subject who donated the effector cells).

Genetic Modification of T Cells and Particularly of Ts Cells and pTs

In a particular embodiment of the invention, the T lymphocytes (general T lymphocyte population) present in the population of mammalian cells or the suppressor T lymphocytes (carrying the THY-1 marker) can be genetically modified so as to express biological products of interest.

The expression “genetically modified” indicates that the cells comprise a nucleic acid molecule which is not naturally present in unmodified T cells, or which is present in said cells when they are not in their natural state (e.g., when they are amplified). The nucleic acid molecule can have been introduced into said cells or into a parent or progenitor cell.

A particular object of the invention thus relates to a method for obtaining or producing suppressor T lymphocytes (and/or the precursors thereof) comprising:
(a) obtaining a mammalian cell population comprising T lymphocytes,
(b) recovering T lymphocytes expressing the THY-1 antigen, and
(c) genetically modifying said T lymphocytes expressing the THY-1 antigen by contacting said lymphocytes with a recombinant nucleic acid molecule.

A particular object of the invention relates to a method for obtaining or producing suppressor T lymphocytes (or the precursors thereof) comprising:
(a) obtaining a mammalian cell population comprising T lymphocytes,
(b) genetically modifying said T lymphocytes by contacting said cell population with a recombinant nucleic acid molecule, and
(c) recovering T lymphocytes expressing the THY-1 antigen.

Several approaches can be used to genetically modify T cells belonging to the mammalian cell population [equivalent to the general T lymphocyte population (effector T cells and Ts cells)] or Ts lymphocytes and pTs, such as for example delivering a gene by means of a virus, naked DNA, physical treatments, and the like. To this end, the nucleic acid is generally incorporated in a vector, such as a recombinant virus, plasmid, phage, episome, artificial chromosome, and the like.

According to a particular embodiment of the invention, the T cells such as defined in the previous paragraph are genetically modified by means of a viral vector (or a recombinant virus). The heterologous nucleic acid is, for example, introduced in a recombinant virus which is then used to infect T lymphocytes. Different types of recombinant virus can be used, in particular recombinant retroviruses or AAV. Preferably, the T lymphocytes are modified by means of a recombinant retrovirus. The use of a retrovirus is particularly appreciated in so far as retroviral infection enables stable integration of the nucleic acid in the cellular genome. This property is particularly important, in so far as amplification of the lymphocytes, whether it be in vitro or in vivo after injection in the subject, requires that the transgene be stably maintained during cell division. Examples of retroviruses that can be used are those from oncovirus, lentivirus and spumavirus families. Specific examples of the oncovirus family are MoMLV, ALV, BLV or MMTV but also RSV, etc. Examples of the lentivirus family include HIV, SIV, FIV, EIAV or CAEV, etc.

Methods by which to construct recombinant retroviruses have been extensively described in the literature (WO 89/07150, WO 90/02806 and WO 94/19478, whose teachings are wholly incorporated in this application). Said methods generally comprise introduction of a retroviral vector containing the transgene into a suitable packaging cell line, followed by recovery of the viruses produced, said viruses comprising the transgene in their genome.

In a particular embodiment of the invention, the recombinant retrovirus comprises the GALV viral envelope (GALV-pseudotyped retrovirus). It has been shown that infection of hematopoietic cells with a recombinant retrovirus is more efficient when the retroviral envelope is from the GALV retrovirus (Gibbon Ape Leukemia Virus). By using said retroviral envelope, a lymphocyte transduction efficiency of more than 95% can be achieved prior to selection of the transduced cells.

The T lymphocytes can be infected with the help of recombinant viruses and by means of various protocols, such as incubation with a viral supernatant, with purified virus, by coculture of T lymphocytes with viral packaging cells, by Transwell techniques, and the like. A particularly efficient method comprising a centrifugation step is described by Movassagh et al. (Movassagh M, Desmyter C, Baillou C, Chapel-Femandes S, Guigon M, Klatzmann D, Lemoine F M., Hum Gene Ther. 1998; 9: 225-234).

Nonviral methods include the use of cationic lipids, polymers, peptides, synthetic agents, and the like. Alternative methods make use of the “gene gun” technique, electric fields, bombardment, precipitation, and the like. When carrying out the invention, it is not necessary for all the Ts cells and pTs to be genetically modified. Hence it is possible to use a T lymphocyte population comprising at least 50%, preferably at least 65%, even more preferably at least 80% of genetically modified lymphocytes. Higher levels (e.g., up to 100%) can be obtained in vitro or ex vivo, for example by using the GALV envelope and/or particular infection conditions (Movassagh et al.) and/or by selecting cells that have effectively been genetically modified. Different selection methods can be used, including the use of antibodies recognizing specific markers present on the surface of modified cells, the use of resistance genes (such as the neomycin resistance gene and the G418 molecule), or the use of compounds toxic to cells not expressing the transgene (e.g., thymidine kinase). Selection is preferably carried out with the help of a marker gene expressing a membrane protein. The presence of said protein allows selection by conventional separation methods such as separation with magnetic beads, the use of columns or flow cytometry.

The nucleic acid used to genetically modify the T cells can be a therapeutic transgene and can code for various active biological products, including polypeptides (e.g., proteins, peptides, etc.), RNAs, and the like. In a preferred embodiment, the nucleic acid codes for a polypeptide exhibiting immunosuppressive activity. In another embodiment, the nucleic acid codes for a polypeptide which is toxic or has conditional toxicity for the cells. Preferred examples include thymidine kinase (which confers toxicity in the presence of nucleoside analogues), such as HSV-1 TK, a cytosine deaminase, gprt, and the like. It can also be a nontoxic polypeptide but one which allows the elimination of injected cells where necessary (such as for example a molecule expressed at the cell membrane and a complement-fixing monoclonal antibody).

Another preferred category of nucleic acids comprises those allowing targeting. They can be nucleic acids coding for a T or B cell receptor or a subunit or functional equivalent of same. For example, the expression in Ts cells of a recombinant TCR specific of an auto-antigen produces Ts cells and pTs that can act more specifically on effector T cells which destroy a tissue in a subject. Other types of biologically active molecules include growth factors, lymphokines (comprising various cytokines which activate Ts cells), immunosuppressive cytokines (like IL-10 or TGF-β), accessory molecules, antigen-presenting molecules, antigen receptors, and the like. The nucleic acid can code for “T-bodies”, i.e., hybrid receptors between T cell receptors and an immunoglobulin. Such “T-bodies” enable the targeting of antigen complexes, for example.

In a preferred manner, the suppressor T lymphocytes (or the precursors thereof) are genetically modified and comprise a recombinant nucleic acid coding for a product displaying conditional toxicity for said cells, such as thymidine kinase. According to another preferred embodiment of the invention, the genetically modified Ts cells and pTs comprise a recombinant nucleic acid molecule coding for a T cell receptor or for a subunit or for a functional equivalent of same.

In some indications, such as allogeneic bone marrow transplantation in particular, one might be led to carry out separate preparations of Ts (and pTs) and effector T lymphocytes, each expressing a different gene coding for a product displaying conditional toxicity, and thereby enabling one or the other of the cell populations to be eliminated.

The nucleic acid which is introduced into the T cells according to the invention typically comprises regulatory sequences, such as a promoter and a polyadenylation sequence, in addition to the coding region.

Compositions

A particular object of this invention is a composition comprising at least one suppressor T lymphocyte according to the invention, e.g., isolated, genetically modified and/or amplified ex vivo, or a population enriched in suppressor T cells such as defined hereinabove or on the contrary a population depleted of suppressor T cells, and a pharmaceutically acceptable adjuvant or medium.

Another particular object of the invention is a composition comprising suppressor T lymphocytes (including the precursors thereof) transduced with a first suicide gene and effector T cells transduced with a second suicide gene, which is different from the first.

The compositions can comprise other cell types, without significantly affecting the therapeutic benefit of said compositions.

According to a preferred embodiment, the cells are conditioned (packaged) in a composition comprising between approximately 10⁵ and 10¹⁰ suppressor T cells according to the pathology to be treated, more generally between 10⁵ and approximately 10⁹ suppressor T cells.

A particular inventive composition comprises a population of THY-1-positive human lymphocytes, displaying suppressor properties with regard to effector T cells.

The medium or adjuvant can be any culture medium, defined medium, aqueous, buffered suspension or solution, optionally supplemented with preservatives. The inventive compositions can be administered by any suitable route, such as intravenous, intra-arterial, subcutaneous, transdermal, and the like. Repeated administrations of said compositions may be given.

Other particular compositions according to the invention comprise a Thy-1-specific ligand coupled or conjugated with an effector molecule, for example a molecule displaying toxicity (conditional or not, for example a TK, ricin toxin, etc.) or a stimulatory activity for T lymphocytes (for example a cytokine, particularly IL-2, IL-7, IL-15, etc.). Said compositions can be used in vivo (or ex vivo) to modulate the repertoire or activity of suppressor T cells in a subject. For instance, the administration of a conjugate comprising a toxic molecule can enable an inactivation or a reduction of suppressor T cell activity in a subject, and therefore an increase in the activity of effector cells. Conversely, the administration of a conjugate comprising an activator molecule can allow to stimulate the activity of suppressor T cells in a subject, and hence to reduce the activity of effector cells. The coupling can be covalent or not.

Other particular inventive compositions comprise a transfection agent coupled with a Thy-1-specific ligand. Said coupling allows to target or promote the interaction between the transfection agent and the Ts cells. The transfection agent can be a viral particle (for example recombinant, defective, attenuated, synthetic, etc.) or a nonviral transfection agent, such as a liposome, cationic lipid, polymer, and the like. The coupling can be covalent or not. Said compositions enable a targeted modification of suppressor T cells in a subject, for example in order to confer novel properties thereto.

Uses

The invention provides cell populations that can be used for the treatment of various pathologies, associated with T cell activity, as indicated earlier. The treatment can be preventive or curative. In addition, the suppressor T cells (including pTs cells), the cell populations enriched in suppressor T cells (including pTs cells) and the compositions of the invention can be used in combination with other active compounds or agents, such as other cell populations, immunosuppressive conditions or molecules, irradiation, gene therapy products, and the like.

The term treatment refers to a reduction in the symptoms or causes of a disease, regression of a disease, delaying of a disease, improving the state of patients, alleviating the patient's suffering, prolonging the patient's survival, and the like.

The suppressor T cells (including pTs cells), the cell populations enriched in suppressor T cells (including pTs cells) and the compositions according to the invention are particularly suited to delaying or preventing graft-versus-host disease (GVHD) in subjects who have undergone allogeneic organ transplantation, particularly of bone marrow (or hematopoietic stem cells or non-hematopoietic stem cells). GVHD and the frequent complications associated with hematopoietic stem cell transplantation are due to the presence of mature donor T cells in the graft. However, removal of said cells prior to grafting results in a failure of the transplant, prolongation of immunosuppression and recurrence of leukemia. Administration of Ts cells according to the invention at the time of transplantation delays or even prevents GVHD. Conversely, it may be advantageous to deplete suppressor T cells (including pTs cells) from a graft in order to increase the reactivity of the injected cells against residual leukemic cells. Said depletion of THY-1 cells can be associated or not with depletion by means of other antibodies such as those specific for CD25 for example.

The suppressor T cells (including pTs cells), the cell populations enriched in suppressor T cells (including pTs cells) and the compositions according to the invention are also suited to the treatment of autoimmune diseases (including chronic inflammatory diseases), such as systemic lupus erythematosus, rheumatoid arthritis, polymyositis, multiple sclerosis, diabetes, atherosclerosis, etc. Autoimmune diseases have an immunologic component as shown by many biological and histological studies. The central factor in such diseases is an inadapted immune response. Furthermore, it is often possible to identify the auto-antigen in such diseases and to define the period during which deleterious T cells are activated. The invention can be used to prevent, treat, reduce or attenuate such pathologies by administering to a subject an efficient amount of suppressor T cells (including pTs cells) in order to suppress or reduce the activity of said deleterious T cells. Repeated administrations may be given if necessary.

The suppressor T cells (including pTs cells), the cell populations enriched in suppressor T cells (including pTs cells) and the compositions according to the invention can also be used for the treatment of infectious diseases and particularly virally-induced immune disorders. The immune response directed against infectious agents can have potentially fatal immunopathological consequences. An example is the response to certain viruses that cause hepatitis. Said viruses replicate in hepatocytes and the destruction of the infected hepatocytes by the immune system induces hepatitis, which sometimes has a fatal outcome. The course of this chronic hepatitis is characterized by biological signs and an abnormal immune response (for example, the presence of anti-DNA antibodies or cryoglobulinemia). The suppressor T cells (including pTs cells), the cell populations enriched in suppressor T cells (including pTs cells) and the compositions according to the invention enable the elimination, suppression or reduction of the active T lymphocytes responsible for the pathology and thereby the reduction of the consequences of virally-induced immune pathologies.

The suppressor T cells (including pTs cells), the cell populations enriched in suppressor T cells (including pTs cells) and the compositions according to the invention can also be used for the treatment or prevention of rejection of transplanted organs such as heart, liver, kidneys, lungs, pancreas, etc. The usual treatment of certain organ disorders consists, when this becomes necessary, in replacing the organ with a healthy organ from a deceased donor (or from a living donor in some cases, or even from a donor from another species). This is also the case for the treatment of insulin-dependent diabetes, by transplanting an insulin-producing organ or cells, such as pancreas or pancreatic islet cells. Although extreme care is taken to select organ donors having maximum compatibility with respect to histocompatibility antigens, the transplanted organ always, except in transplants between homozygous twins, induces the development of an immune response directed against the antigens specifically expressed by said organ. Despite immunosuppressive treatments, this reaction often results in rejection of the organ transplant (this is the leading cause of failure of allogeneic transplantation). With the exception of certain superacute or acute rejections which involve mainly the humoral response, organ transplant rejection is, in the majority of cases, mediated primarily by effector T lymphocytes.

The invention now makes it possible to envision a treatment (e.g., the reduction or the postponement) of organ rejection with the help of suppressor T cells (including pTs cells). Said cells can be prepared from the patient's cells, stimulated with donor antigens and re-administered to the patient, before or during organ transplantation. Repeated administrations may be given if necessary. This approach is particularly adapted to the treatment of diabetes, i.e., in order to reduce, delay or prevent the rejection of transplanted insulin-producing cells, tissues or organs (particularly pancreatic islet cells). Typically, the Ts cells are amplified and activated by culturing them in the presence of auto-antigens arising from the donor tissue. Said cells can be produced for example by culture in the presence of dendritic cells autologous with respect to the graft. Said amplified and activated suppressor T cells (including pTs cells) can be injected in the patient before, during and/or after the organ transplantation, thereby reducing the destructive activity of effector T cells.

The suppressor T cells (including pTs cells), the cell populations enriched in suppressor T cells (including pTs cells) and the compositions according to the invention are also suited to the treatment of allergies, which are mediated by immune responses directed against particular antigens called allergens. By administering to patients the suppressor T cells (including pTs cells), optionally activated ex vivo with said allergens, it is possible to reduce these deleterious immune responses.

Another object of the invention relates to a method for reducing the activity (and/or the quantity) of effector T lymphocytes in a mammalian host, said method comprising administering to the mammal suppressor T lymphocytes (or the precursors thereof) according to the invention compatible with said mammalian host, preferably autologous.

Reduction of suppressor T lymphocytes (or the precursors thereof) may also be desired (for example in the context of cancer treatment). Several treatment modalities can be used.

A first approach consists in ex vivo preparation of cells having an activity (for example anti-cancer), depleted of suppressor T cells (including pTs cells). Said depletion can be carried out ex vivo according to an inventive method such as described hereinabove. The depletion can be carried out ex vivo without a preliminary culture phase and/or after a culture phase in a medium containing N-acetyl cysteine which reduces the proliferation of Ts lymphocytes (including pTs cells) (see FIG. 1). The treatment then consists in re-administering to the patient a population of T lymphocytes or a composition comprising such population depleted of suppressor T cells (including pTs cells), and having or not having been activated ex vivo. Said treatment can be accompanied by one or more vaccinations (for example anti-tumoral), combined or not with chemotherapy and/or radiotherapy, in a patient who optionally received conditioning. In particular, said conditioning can comprise lymphoablative, myeloablative treatments or not, intended to eliminate T lymphocytes, particularly T lymphocytes in division, and which comprise suppressor T cells (including pTs cells) responsible for the absence of an effective immune response (such as for example Ts which prevent the development of an effective anti-tumor response).

Another modality consists in depleting suppressor T cells in vivo by using a ligand and any appropriate toxic molecule or activity (such as radioactivity or a toxin for example).

The treatment of all these pathologies can also be carried out by in vivo modulation (suppression or activation) of suppressor T cells (including pTs cells) with the help of any molecules having said activties, and in particular anti-THY-1 antibodies, or any molecules modulating the activity of suppressor T cells (including pTs cells) the discovery of which results from knowledge of the transcriptome and proteome of suppressor T cells (including pTs cells).

The suppressor T lymphocytes (including pTs cells) can also be activated in vivo, for example by Thy-1 ligands coupled to lymphocyte activation molecules (IL-2, IL-10 for example). The treatment can then be administered systemically (intravenous for example) or at the site where the action is desired (in the synovial fluid for the treatment of rheumatoid arthritis for example).

A particular object of the invention corresponds to the use, in the context of vaccination, of a suppressor T cell (including pTs cells), a cell population enriched in suppressor T cells (including pTs cells) or a composition according to the invention.

Different routes of administration and protocols can be implemented in the scope of the invention. They can be adapted by those skilled in the art according to the disease to be treated. Generally, systemic or local administrations can be envisioned and use the intravenous, intra-arterial, intraperitoneal, intramuscular or subcutaneous route, etc. The cells can be injected during the surgical operation or by any appropriate means, for example with the help of a syringe. To control diseases like GVHD, graft-versus-infection effects (GVI) or graft-versus-leukemia effects (GVL) or else rejection of a transplanted organ, the cell composition can be administered before, during or after the bone marrow (or organ) transplantation. Furthermore, additional administrations can be given after the transplantation, so as to prevent or postpone the pathology.

It is understood that the invention is not limited to the specific embodiments described hereinabove, but also encompasses variants that are part of the normal knowledge of those skilled in the art.

### LEGENDS OF FIGURES

FIG. 1: Effect of N-acetyl cysteine on the preferential expansion or not of human CD90+ T lymphocytes

Purified CD3+ T lymphocytes were cultured in RPMI medium supplemented with 10% human serum, interleukin-2, anti-CD3 antibodies and in the presence or absence of N-acetyl cysteine (NAC). The percentage of CD3+ T cells expressing the CD90 marker over time was determined by flow cytometry.

FIG. 2: Effect of dendritic cells on the preferential expansion or not of human CD4+/CD90+ T lymphocytes.

Dendritic cells (DC) derived from CD34+ cells and enriched for the CD1a marker (langerhans DC) or the CD14 marker (interstitial DC) were cultured with allogeneic T lymphocytes in a 1:5 ratio for five days. The percentage of CD3+ T cells expressing the CD90 marker over time was determined by flow cytometry.

FIG. 3: Expression of CD25 and CD90 antigens by human CD4+ (A) and CD8+ (B) T lymphocytes.

T cells were labelled with antibodies recognizing the CD4, CD8, CD25 and CD90 antigens. The expression of these different markers was studied by flow cytometry.

FIG. 4: CD4+/CD90+ and CD8+/CD90+ T lymphocytes have a suppressor function Purified CD4/CD90+, CD4/CD25++ and CD8/CD90+ populations were irradiated with 15 grays, cultured for four days at an equivalent ratio with autologous T lymphocytes depleted of CD25 cells (CD25−) stimulated (A) with a mixture of OKT3/CD28 antibodies, (B) with EBV-transformed allogeneic B lymphocytes, (C) with allogeneic dendritic cells (DC). CD25− cells stimulated alone under the same conditions were used as positive control. Proliferation was evaluated after four days by tritiated thymidine incorporation.

FIG. 5: Expression of the FoxP3 gene by CD4+/CD90+ and CD8+/CD90+ T lymphocytes

Expression of the CD4, IL-10, CTLA-4 and FoxP3 genes was studied by RT-PCR on the different purified cell populations CD4+/CD25−, CD4+/CD25++, CD4+/CD90+, CD8+/CD90+.

FIG. 6: CD90 identifies precursors of CD4/CD25++ suppressor lymphocytes CD4/CD90+, CD4/CD25+, CD4/CD25++ cell populations were highly purified by cell sorting, cultured in RPMI medium containing human serum AB, IL-2 and a mixture of OKT3/CD28 antibodies for seven days. The CD90 and CD25 markers were analyzed at different time points during culture. At day 7, the populations were enriched by cell sorting, irradiated with 15 grays and tested for suppressor activity by coculturing them at an equivalent ratio with allogeneic T lymphocytes doubly depleted of CD25 and CD90 cells (CD25−/CD90−) stimulated with a mixture of OKT3/CD28 antibodies. The numbers indicate the percent inhibition of proliferation in comparison with the control (CD25−/CD90− cells cultured and stimulated alone).

FIG. 7: CD90 identifies CD4+/CD90+ and CD8+/CD90+ suppressor lymphocytes after six days of culture

CD3 T lymphocytes were cultured in the presence of IL-2 and a mixture of OKT3/CD28 antibodies for six days. Analysis of the CD25 and CD90 markers on the CD4 and CD8 populations allowed a determination of the percentages of CD25+ and CD90+ cells (A). CD4/CD25++, CD4/CD90+ and CD8/CD90+ cells were then sorted, irradiated and tested for suppressor activity by coculturing them at an equivalent ratio with allogeneic T lymphocytes depleted of CD25 cells (CD25−) stimulated with a mixture of OKT3/CD28 antibodies (B). Proliferation was evaluated after four days by tritiated thymidine incorporation.

FIG. 8: CD90 enables the identification in a CD25−/CD90− T lymphocyte culture of the appearance of suppressor lymphocytes and precursor cells

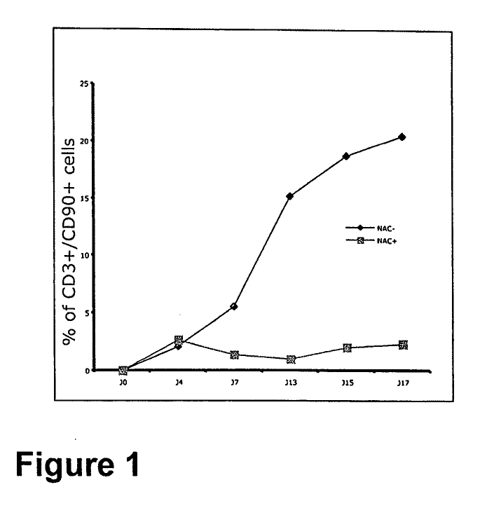

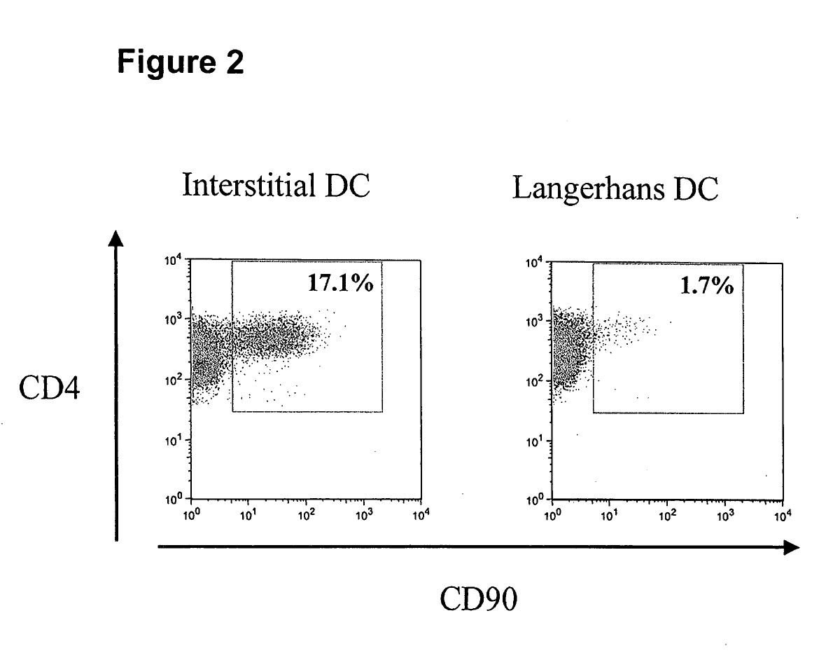

T lymphocytes doubly depleted of CD25 and CD90 cells (CD25−/CD90−) were cultured in the presence of IL-2 and a mixture of OKT3/CD28 antibodies for seven days. The CD90 and CD25 markers were analyzed at different time points during culture. At day 7, the CD25+/CD90+ population was enriched by cell sorting, irradiated with 15 grays and tested for suppressor activity by coculturing them at an equivalent ratio with allogeneic T lymphocytes doubly depleted of CD25 and CD90 cells (CD25−/CD90−) stimulated with a mixture of OKT3/CD28 antibodies. The numbers indicate the percent inhibition of proliferation in comparison with the control (CD25−/CD90− cells cultured and stimulated alone).

FIG. 9: Use of the CD90 marker in human pathology: example of multiple sclerosis Mononuclear cells obtained on a Ficoll gradient from healthy donors (n=6), patients with multiple sclerosis in the chronic phase (multiple sclerosis MS, n=5), and patients with multiple sclerosis in the acute phase (acute MS, n=3) were labelled with CD4, CD25, CD90 monoclonal antibodies. The percentage of CD4+ T cells expressing the CD25 and CD90 marker was studied by flow cytometry.

FIG. 10: Use of the CD90 marker in human pathology: example of a patient with IPEX syndrome.

Mononuclear cells obtained on a Ficoll gradient from healthy donors and from patients with IPEX syndrome confirmed by sequencing the FoXP3 gene were labelled with CD4, CD25, CD90 monoclonal antibodies. The percentage of CD4+ T cells expressing the CD25 and CD90 marker was studied by flow cytometry. The results for an IPEX patient are shown.

### EXAMPLES

1. The CD90 Marker is Expressed by Human CD4+/CD25+ T Lymphocytes and by Human CD8+/CD25+ T Lymphocytes

To study the expression of the CD90 marker comparatively with CD25 in CD4+ and CD8+ T lymphocyte populations, adult peripheral blood mononuclear cells were obtained on a Ficoll gradient then labelled with the following antibodies directly bound to fluorochromes: anti-CD4, anti-CD8, anti-CD25 and anti-CD90. For immunophenotypic analysis, the cells were analyzed by flow cytometry (FACscalibur), and events were reanalyzed with Cellquest and FlowJo software.

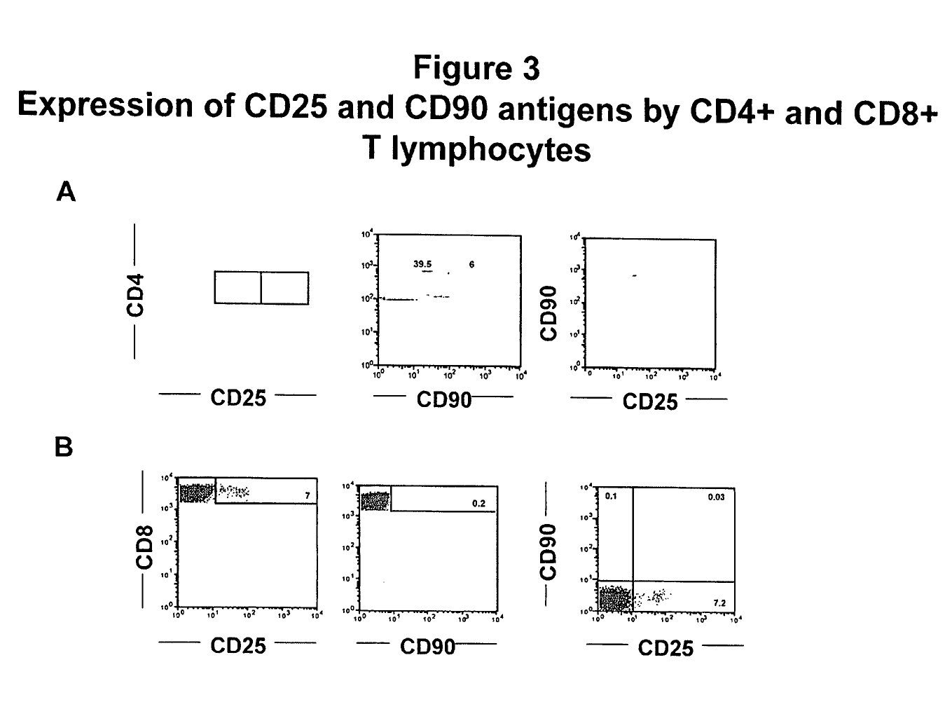

FIG. 3A illustrates the expression of the CD25 and CD90 markers in CD4+ T lymphocytes and the co-expression of CD25 and CD90 in CD4+ cells. It can be seen that 6% and 1.2% of CD4+ lymphocytes expressed the CD25 and CD90 markers, respectively. The majority (>80%) of CD4+/CD90+ cells showed intermediate expression of CD25+ whereas approximately 5% and 15% of CD4+/CD90+ cells were respectively CD25++ and CD25−.

FIG. 3B shows the expression of the CD25 and CD90 markers in CD8+ T lymphocytes and the co-expression of CD25 and CD90 in CD8+ cells. It can be seen that 7% and 0.2% of CD8+ lymphocytes expressed the CD25 and CD90 markers, respectively. It should be noted that, in contrast to CD4+ cells, no CD8+ cells strongly expressed CD25. The majority (75%) of CD8+/CD90+ cells were CD25− whereas approximately 25% of CD8+/CD90− cells were CD25+.

2. The CD90 Marker Identifies Human Suppressor T Lymphocytes in CD4+ and CD8+ Populations.

To demonstrate that CD4+/CD90+ cells have a suppressor function, autologous CD4+ T lymphocytes depleted of CD4+/CD25+ cells (CD25−) were stimulated with a mixture of OKT3/CD28 antibodies previously immobilized on the bottom of the well. CD25− cells were cultured alone or in the presence of an equal number of CD4+/CD90+ or CD4+/CD25+ cells (positive control) for four days, after which proliferation was evaluated by tritiated thymidine incorporation as measured in a β counter. In these experiments, the CD4+/CD90+ or CD4+/CD25+ cells were previously irradiated with 15 grays. The results are expressed in cpm. The percent inhibition was calculated according to the formula: % inhibition=No. cpm (1−No. cpm (CD25−+Ts)/No. cpm (CD25−)×100.

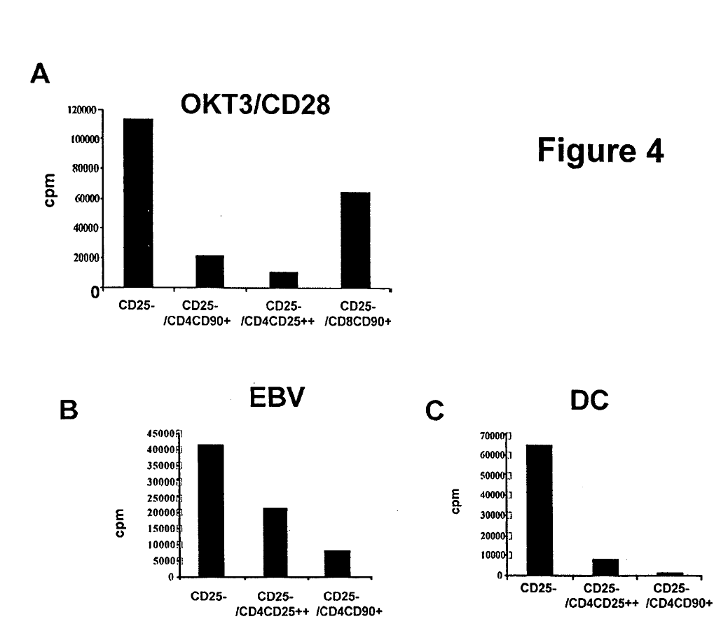

FIG. 4A shows that CD4+/CD90+ and CD4+/CD25++ populations inhibit the proliferation of autologous CD25− T lymphocytes. The results indicate that CD4+/CD90+ cells inhibited CD25− cell proliferation by more than 75%, thereby illustrating their suppressor function.

Experiments were also carried out using allogeneic EBV cells or allogeneic dendritic cells (DC) to stimulate the proliferation of CD25− cells. By adding CD4+/CD90+ or CD4+/CD25+ cells, it was shown that said cells exert a suppressor action on CD25− cell proliferation, as illustrated in FIGS. 4B and 4C.

To demonstrate that CD8+/CD90+ cells have a suppressor function, said cells were placed in the presence of an equal number of CD25− cells stimulated with a mixture of OKT3/CD28 antibodies and cell proliferation was evaluated four days later by tritiated thymidine incorporation. The results in FIG. 4A show that the CD8+/CD90+ population exerted a suppressor function on the proliferation of CD25− cells.

3. CD4+/CD90+ and CD8+/CD90+ Lymphocytes Express Foxp3, CTLA4 and TGFβ

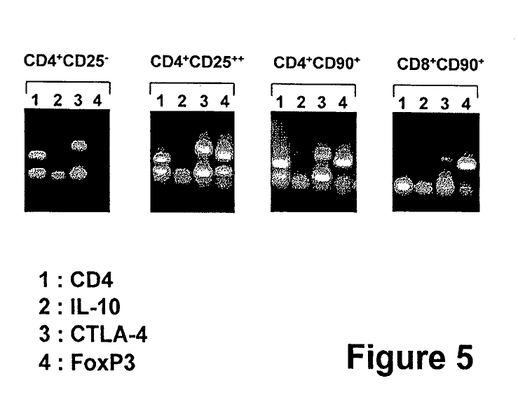

The expression of the Foxp3, CTLA4, CD4, CD25, TGFβ genes was analyzed by nested PCR following reverse transcription of RNA extracted from 1000 or 5000 cells from the different lymphocyte populations under study. The results in FIG. 5 show that CD4+/90+ and CD8+/CD90+ lymphocytes expressed Foxp3, CTLA4 and TGFβ, like the CD4+/CD25++ cells.

4. CD90 Enables the Identification of a Population of CD4+/CD25+ Lymphocyte Precursor Cells

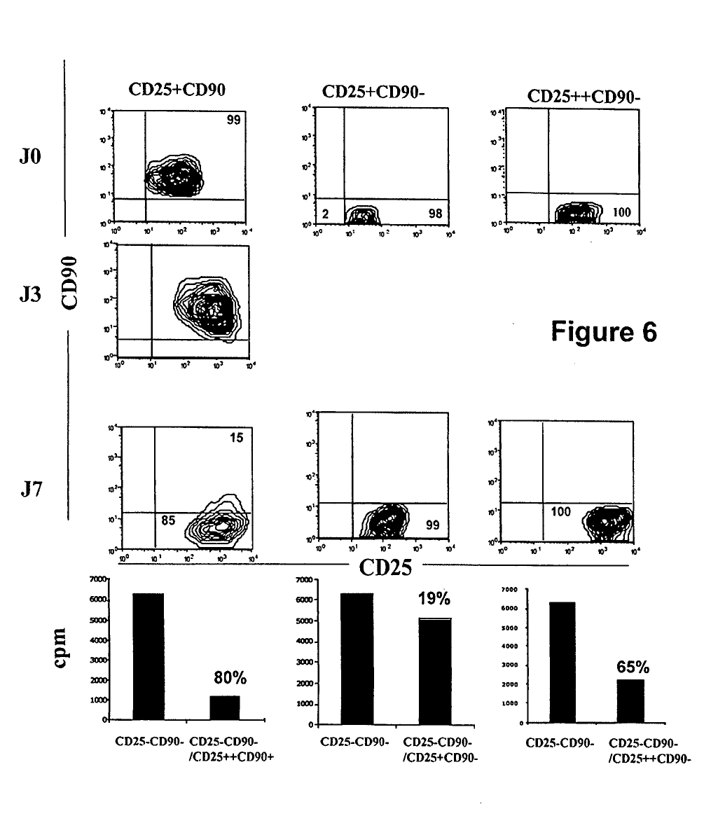

To determine whether the CD4+/CD90+ population is related to CD4+/CD25++ cells, CD4+/CD90+ cells were highly purified by flow cytometry (purity>98%) and cultured in liquid medium in the presence of a mixture of OKT3/CD28 antibodies and interleukin 2. The cells were sequentially analyzed by flow cytometry between days 1 and 7 of culture for the markers CD4/CD90 and CD4/CD25. The sorted CD4+/CD25++ and CD4+/CD25+/CD90− populations which respectively represent conventional suppressor T lymphocytes and activated T lymphocytes were cultured in parallel in the same conditions. The results in FIG. 6 indicate that CD4+/CD90+ cells gradually lost the CD90 marker and became highly positive for the CD25 marker. The immunophenotypic evolution of CD4+/CD25++ cells, which initially were CD90−, indicates that this population even more strongly overexpressed the CD25 marker after several days of culture without acquiring the CD90 marker. The CD4+/CD25+/CD90− population strongly acquired the CD25 marker but not CD90. To study the suppressor function of these different populations after culture, the cells were sorted, irradiated with 15 grays and placed in the presence of allogeneic CD25− cells.

The results indicate that 1) CD4+/CD90+ cells are able to give rise to CD4+/CD25++cells having suppressor activity; 2) the CD4+/CD25++ cells conserve their suppressor activity; 3) CD4+/CD25+/CD90− cells give rise to CD25++ cells without suppressor activity. These results show that CD4+/CD90+ cells can give rise to CD4+/CD25++ suppressor cells and can be considered precursor cells (pTs) of suppressor lymphocytes (Ts).

5. CD90 Enables the Identification of CD4+/CD90+ and CD8+/CD90+Suppressor Lymphocytes After Culture.

To determine whether the CD90 marker, in contrast to the CD25 marker which is also expressed in activated T lymphocytes, enables the identification of suppressor T lymphocytes after activation and culture of T lymphocytes, RPMI 1640 liquid medium containing 10% human serum AB and 5 mcg of OKT3 antibody was used to culture total T lymphocytes of which the CD4+/CD90+, CD8+/CD90+ and CD4+/CD25+ populations were purified by flow cytometry after six days of culture. After culture, the different cell types were analyzed by cytometry and tested for suppressor activity.

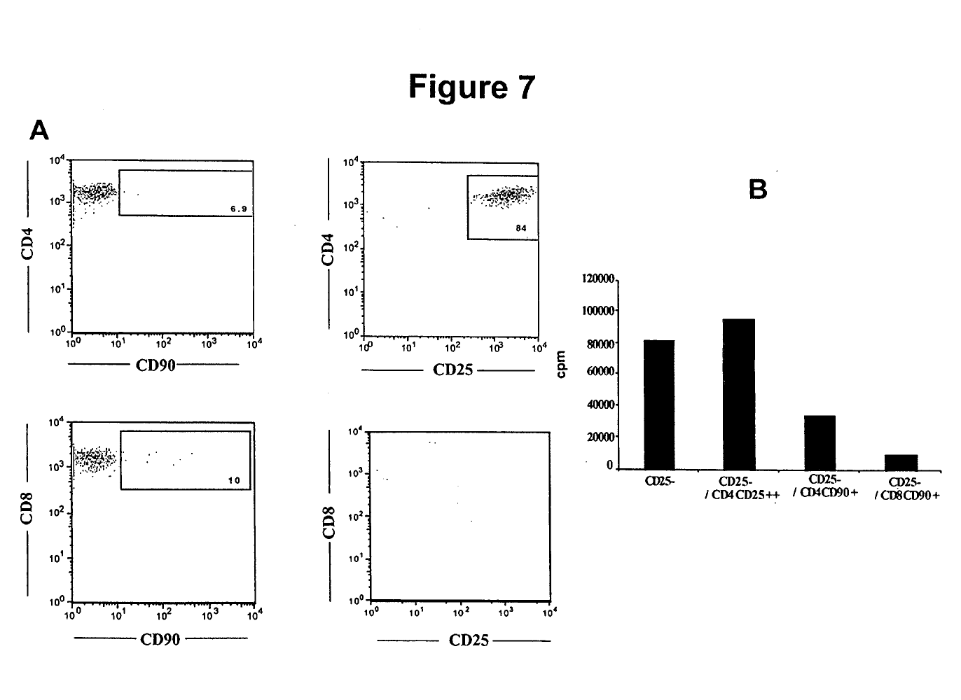

FIG. 7A shows the expression of the CD25 and CD90 markers in CD4+ and CD8+ lymphocytes after six days of culture. It can be seen that 6.9% and 10% of CD4+ and CD8+ T lymphocytes, respectively, expressed the CD90 marker, while 84% and 94.3% of CD4+ and CD8+ T lymphocytes expressed the CD25 marker.

FIG. 7B shows that the CD4+/CD90+ and CD8+/CD90+ populations inhibited the proliferation of autologous CD25− T lymphocytes whereas CD4+/CD25+ lymphocytes no longer had a suppressor effect on CD25− cells after culture. These findings indicate that, in contrast to CD25, the CD90 marker is specific of suppressor populations within cultured CD4+ and CD8+ T lymphocytes.

6. CD90 Identifies the Appearance of Suppressor Lymphocytes and Precursor Cells from Cultured CD25−/CD90− T Lymphocytes.

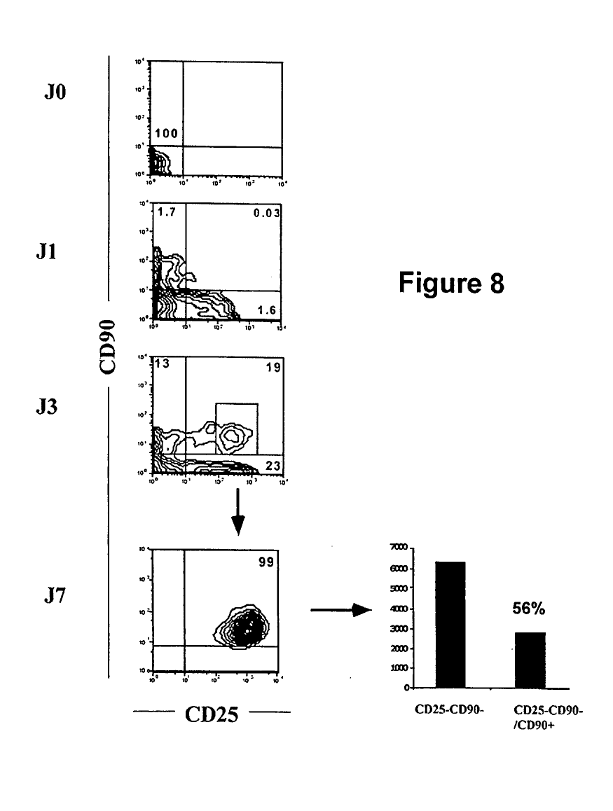

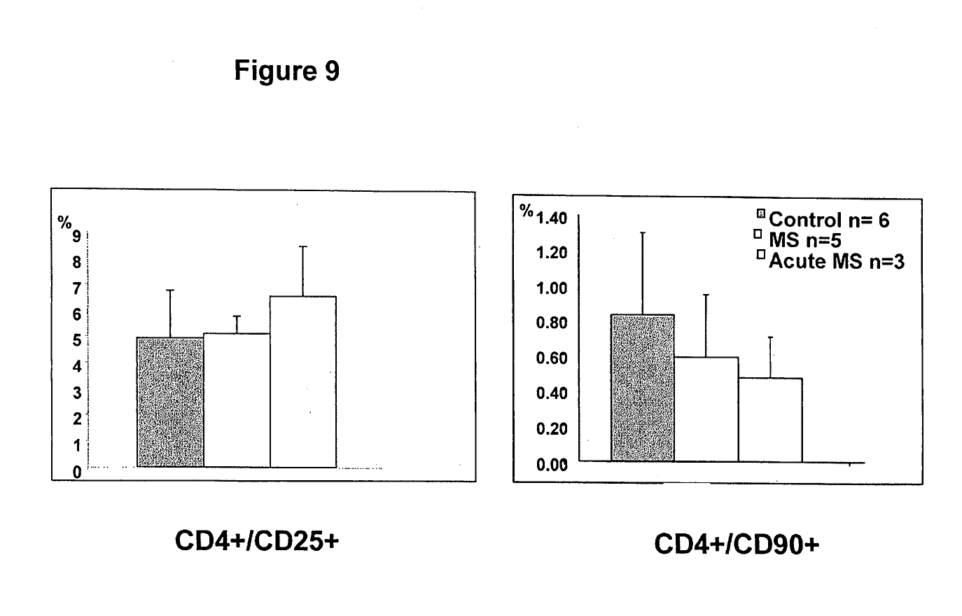

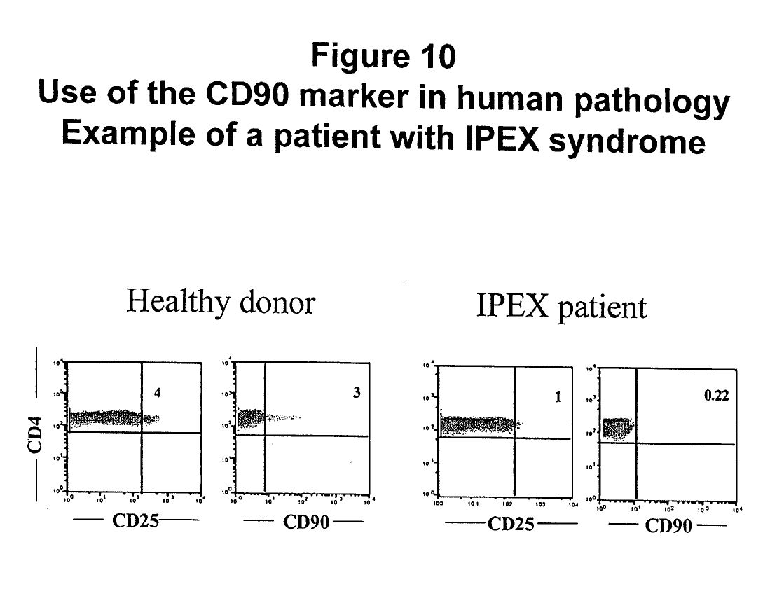

To determine whether, after activation and culture of T lymphocytes doubly depleted of CD25 and CD90 cells (CD25−/CD90− cells), the CD90 marker still allows the identification of suppressor T lymphocytes, CD25−/CD90− T lymphocytes were cultured in RPMI 1640 liquid medium containing 10% human serum AB and 5 mcg of OKT3 antibody. The CD25 and CD90 markers were analyzed at different time points during culture by flow cytometry. The results in FIG. 8 reveal the appearance of two routes of differentiation starting from 24 hours of culture. Thus, it was possible to detect CD90+/CD25− cells concomitantly with the appearance of CD25+/CD90− cells. After 2-3 days, the CD90+ cells became CD90+/CD25++ whereas the CD25+/CD90− cells became CD25++/CD90−. The sorting and functional study of CD90+/CD25++ cells showed that these cells inhibited the proliferation of autologous CD25−/CD90− T lymphocytes stimulated by OKT3/CD28 antibodies. These findings indicate that it is possible to generate suppressor T lymphocytes that can be identified by the CD90 marker starting from CD25−/CD90− T lymphocytes.

7. Identification and Diagnostic Monitoring.

We have shown that patients presenting with autoimmune complications of hepatitis C have a deficit of CD4+/CD25+ lymphocytes (Boyer et al., Blood, in press). Other authors report a similar deficit in type 1 diabetes. The diagnosis and the biological and clinical monitoring of these pathologies will be more specific by monitoring Ts cells by means of CD90 labelling, which in particular identifies a Ts precursor population. Said monitoring will be all the more important for diseases which progress by flare-ups, such as rheumatoid arthritis or multiple sclerosis for example. The choice and the time of the therapeutic intervention, which in particular can be an injection of Ts cells, will be defined by monitoring Ts cells through the CD90 marker. The identification of Ts cells is carried out in any biological fluid of interest (blood, CSF, synovial fluid for example) or in any tissue or organ of interest (tumor, transplanted organ, etc.).

By way of example, patients with multiple sclerosis in the chronic phase (MS) and in the acute phase (acute MS) were studied. FIG. 9 reveals an increase in CD4+/CD25+ T lymphocytes (activated T lymphocytes) and in contrast a decrease in CD4+/CD90+ cells during acute MS as compared with the chronic phase or the control group. These findings illustrate the interest of the CD90 marker for evaluating the reduction in suppressor T lymphocytes during an acute episode of an autoimmune disease by distinguishing them in particular from activated T lymphocytes.

By way of example, patients with IPEX syndrome were studied. FIG. 10 shows that CD4+/CD90+ cells were virtually absent from the blood of an IPEX patient as compared with a healthy donor whereas the use of the CD25 marker which also recognizes activated T lymphocytes was unable to reveal this difference.

8. Therapeutic Injection of Ts Cells for Control of GVHD.

We have shown that Ts cells play an important role in controlling GVHD and that it is possible to prepare specific Ts by allo-activation (Cohen et al., JEM 2003, Trenado et al., JCI 2003). For these applications, the Ts can be obtained from blood, cord blood, bone marrow, any tissue containing T lymphocytes. In these applications, the Ts may be genetically modified or not.

9. Therapeutic Injection of Ts Cells for Control of MS.

Ts cells were obtained from the patient or from a compatible donor, preferably geno-identical. They were purified by immunomagnetic beads, flow cytometry, by adhesion to a solid support coated with specific antibodies (panning) and optionally frozen. The patient's Ts cell count was monitored. In the event of appearance of clinical signs indicating the onset of a flare-up or if there was a decrease in the Ts count, the patient received an injection of Ts cells prepared for that occasion, or prepared previously.

10. Ablation of Ts Cells for the Treatment of Tumors.

We have shown that Ts cells prevent the mounting of an efficient anti-tumoral immune response. Depletion of said cells enables these responses to develop. Furthermore, the preparation of anti-tumoral T lymphocytes ex vivo runs the risk of contamination by Ts cells.

The principle of the treatment is therefore to eliminate Ts in vivo, in particular with the help of a CD90 ligand coupled with a toxin. It may also be a matter of depleting the entire set of T lymphocytes by classical treatments (anti-lymphocyte serum, anti-CD3, Campath antibodies, irradiation, etc., for example). Said treatment can be completed by administering lymphocytes activated ex vivo against tumor antigens after being depleted of Ts cells.

1. Method for obtaining, preparing or producing human suppressor T lymphocytes and/or the precursors thereof, comprising a step of selection, separation or isolation in vitro or ex vivo of human T lymphocytes expressing the THY-1 molecule.

2. Method according to claim 1, comprising:
(a) obtaining a cell population of human origin comprising T lymphocytes, and
(b) recovering T lymphocytes expressing the THY-1 antigen.

3. Method according to claim 1, wherein the step (b) is preceded or followed by a step of amplification of T lymphocytes.

4. The method of claim 1 wherein the T lymphocytes expressing the THY-1 antigen are selected, separated, isolated or recovered by means of a ligand specific of THY-1.

5. Method according to claim 4, wherein the specific ligand is an antibody specific of THY-1 or a fragment or derivative of said antibody having substantially the same antigenic specificity.

6. Method according to claim 5, wherein the specific ligand is a monoclonal or polyclonal antibody specific of THY-1.

7. Method according to claim 5, wherein the specific ligand is a polyfunctional, monocatenary or multimeric antibody, specific of THY-1.

8. Method according to claim 4, wherein the specific ligand is an aptamer.

9. Method according to claim 4, wherein the ligand is immobilized on a support or placed in solution.

10. Method according to claim 9, wherein the support is a column or a bead, preferably a magnetic bead.

11. Method according to claim 4, wherein the ligand is labelled.

12. Method according to claim 11, wherein the labelling is carried out by means of a fluorescent, radioactive, luminescent, phosphorescent, chemical or enzymatic detection label.

13. Method according to claim 1, wherein the step of recovery, selection or isolation is carried out by flow cytometry, affinity chromatography, FACS, MACS or D/MACS.

14. Method according to claim 2, wherein the cell population comes from a tissue selected in the group consisting of bone marrow, spleen, liver, thymus, blood which has or has not been previously enriched in T lymphocytes, umbilical cord blood, fetal, infant or adult peripheral blood, a tumor, a site of inflammation, a transplanted organ or a cell culture established with one or another of said tissues.

15. Method for identifying and/or quantifying human suppressor T lymphocytes in a cell population, comprising exposing said cell population to a ligand specific of THY-1 and determining and/or quantifying the formation of a complex between the ligand and the cells, the formation of said complexes indicating the presence and/or the quantity of suppressor T lymphocytes in the cell population.

16. Method for producing a pharmaceutical composition, comprising:
(a) obtaining a biological sample comprising human T lymphocytes,
(b) selecting T lymphocytes expressing the THY-1 antigen in said biological sample, and
(c) conditioning said T lymphocytes expressing the THY-1 antigen in a pharmaceutically acceptable adjuvant or medium.

17. Method for producing a pharmaceutical composition, comprising:
(a) obtaining a biological sample comprising human T lymphocytes,
(b) depleting T lymphocytes expressing the THY-1 antigen from said biological sample, and
(c) conditioning said T lymphocytes not expressing the THY-1 antigen in a pharmaceutically acceptable adjuvant or medium.

18-28. (canceled)
